(19) United States
(12) Patent Application Publication
(10) Pub. No.: US 2004/0058016 A1
(43) Pub. Date: Mar. 25, 2004

(54) Lower alcohol-insoluble extract of hovenia dulcis var koreana nakai, a polysaccharide isolated therefrom and an antihepatotoxic composition containing same

(76) Inventors: Chun-Soo Na, Suwon-si (KR); Nam-Chul Jung, Suwon-si (KR)

Correspondence Address: David A Einhorn, Anderson Kill & Olick, 1251 Avenue of the Americas, New York, NY, 10020, US

(21) Appl. No.: 10/467,097

(22) PCT Filed: Jan. 31, 2002
(86) PCT No.: PCT/KR02/00142

(30) Foreign Application Priority Data
Jan. 31, 2001 (KR) 2001/4633

(51) Int. Cl. A61K 31/715; A61K 35/78; C08B 37/00

(52) U.S. Cl. 424/725; 514/54; 536/123

(57) ABSTRACT

A pharmaceutical composition and health care food comprise a lower alcohol-insoluble extract of Hovenia dulcis var. Koreana NAKAI or a polysaccharide isolated therefrom having a potent hepatoprotective activity.

### FIELD OF THE INVETION

The present invention relates to a pharmaceutical composition containing a lower alcohol-insoluble extract of Hovenia dulcis var. Koreana NAKAI and a polysaccharide isolated therefrom having a hepatoprotective activity.

### BACKGROUND OF THE INVENTION

Hepatitis afflicts increasing numbers of population, and due to the lack of therapeutically effective drugs, it usually progresses to chronic hepatitis, liver cirrhosis or cancer. Various types of hepatitis may be developed when a patient is exposed to, e.g., stress, excessive consumption of alcohol, and/or hepatotoxic substances.

Exemplary hepatotoxic substances are CCl₄, D-galactosamine, lipopolysaccharide (LPS), bromobenzene and the aldehyde such as acetaldehyde which is an intermediate in the metabolic pathway of alcohol. Accordingly, there have been many attempts to find new drugs which can protect the liver from such hepatotoxic substances or restore the liver function damaged thereby.

For example, triterpene glycoside components isolated from the seed and fruit of Hovenia dulcis Thunb were known to inhibit the release of histamine and the absorption of alcohol in human body (Yoshikawa, K. T. et al., (1995) Chem. Pharm. Bull. Tokyo, 43(3), pp532-534); and the fruit of Hovenia dulcis Thunb were found to inhibit liver damage inducible carbon tetrachloride or D-galactosamine/lipopolysaccharide (Hase K. et al., (1997) Chem. Pharm. Bull. Tokyo, 20(4), pp381-385). The above and other prior art literatures describe pharmacological activities of extract of the fruits or seeds of Hovenia dulcis Thunb. var. tomentella Makino (Japanese species) and Hovenia dulcis Thunb. (Chinese species); however there have been no reports directed to the use of Hovenia dulcis Thunb. var. Koreana NAKAI (Korean species) as a source of new pharmacologically active agents.

Hovenia dulcis Thunb. var. Koreana NAKAI is a rare deciduous plant species belonging to the Ramnaceae family and is indigenous to Korea, distributed in the southern parts of Kangwon Province. Hovenia dulcis Thunb. var. Koreana NAKAI is distinctly different from Hovenia dulcis Thunb. var tomentella Makino and Hovenia dulcis Thunb in that while both the Chinese and Japanese species exhibit bright green petals, the petal of the Korean species is white, and also in that the size of fruit-peduncle and the seed shape are not the same. Therefore, Hovenia dulcis Thunb. var. Koreana NAKAI has been classified as a different species (Uehara K. I., (1960) JUMOKUDAITSUSETSU, Yumei Press. 2^{nd} Ed. pp 1072-1074), and its fruit and seed have been used for quenching thirst and treating nausea (Chung, B. S. et al., (1998) DOHAEHYANGYAKDAISAJEON (Plant part), Youngrim Press, pp291-292).

### SUMMARY OF THE INVENTION

Accordingly, it is an object of the present invention to provide a pharmacologically active substance extracted from Hovenia dulcis Thunb. var. Koreana NAKA.

Another object of the present invention is to provide a method for isolating said substance from Hovenia dulcis Thunb. var. Koreana NAKAI.

An additional object of the present invention is to provide a pharmaceutical composition for inhibiting alcohol dehydrogenase and lactic acid dehydrogenase, comprising a pharmaceutically acceptable carrier and the above-described substance isolated from Hovenia dulcis Thunb. var. Koreana NAKAI.

A further object of the present invention is to provide a pharmaceutical composition for preventing or inhibiting a liver disease, comprising a pharmacologically acceptable carrier and a polysaccharide isolated from Hovenia dulcis Thunb. var. Koreana NAKAI.

A still further object of the present invention is to provide a health care food comprising said substance and/or the polysaccharide derived from Hovenia dulcis Thunb. var. Koreana NAKAI.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic procedure for preparing a methanol-insoluble fraction of an extract of the fruit-peduncle of Hovenia dulcis Thunb. var. Koreana NAKAI, and a polysaccharide isolated therefrom;

FIG. 2: the melting point and melting enthalpy spectrum of the methanol-insoluble fraction;

FIG. 3: a MALLS spectrum of the methanol-insoluble fraction;

FIG. 4: an IR spectrum of the methanol-insoluble fraction;

FIG. 5: a UV spectrum of the methanol-insoluble fraction;

FIG. 6: a GC spectrum of the methanol-insoluble fraction;

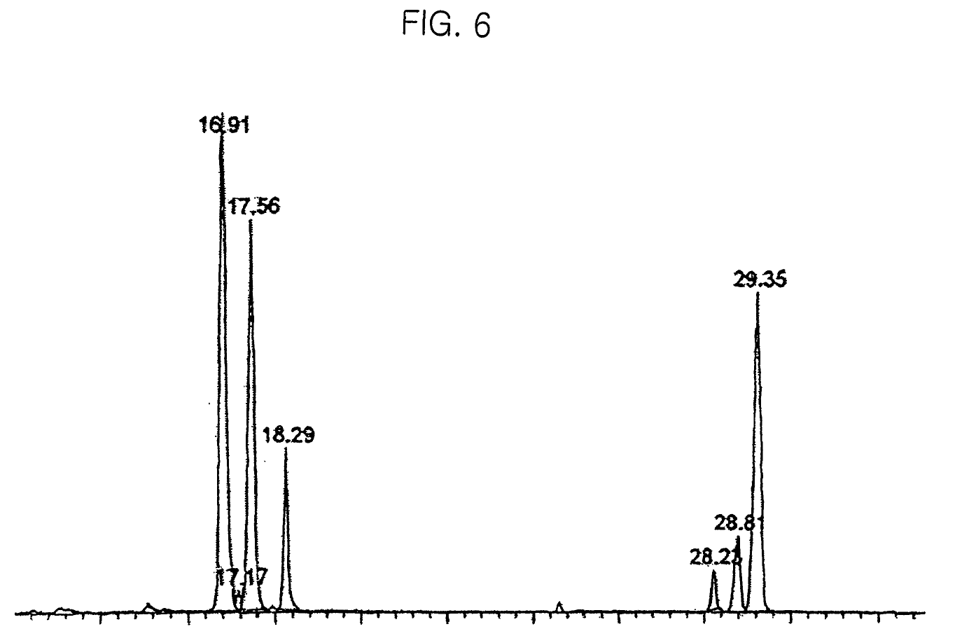

FIG. 7: a MALLS spectrum of the methanol-insoluble polysaccharide;

FIG. 8: an IR spectrum of the methanol-insoluble fraction;

FIG. 9: a NMR spectrum of the methanol-insoluble fraction;

FIG. 10: an in vitro protein synthesis activity of the methanol-insoluble fraction on a carbon tetrachloride-induced liver slice culture;

FIG. 11: an in vitro protein synthesis activity of the methanol-insoluble fraction on a galactosamine/LPS-induced liver slice culture;

FIG. 12: an in vitro protein synthesis activity of the methanol-insoluble fraction on a bromobenzene-induced liver slice culture;

FIG. 13: an LDH release inhibitory activity of the methanol-insoluble fraction on the bromobenzene-induced liver slice culture; and

FIG. 14: an in vitro protein synthesis activity of the polysaccharide shown in FIG. 7 on the bromobenzene-induced liver slice culture.

### DETAILED DESCRIPTION OF THE INVENTION

In accordance with one aspect of the present invention, there is provided a lower alcohol-insoluble fraction obtained by treating a hot-water extract of dried Hovenia dulcis Thunb. var. Koreana NAKAI with an lower alcohol, In accordance with another aspect of the present invention, there is also provided a polysaccharide having a potent hepatoprotective activity, which is isolated from said lower alcohol-insoluble fraction.

As used herein, “lower alcohol” means C₁˜C₄ alcohol and exemplary lower alcohol may include methanol, ethanol and butanol.

The lower alcohol-insoluble fraction of an extract of Hovenia dulcis Thunb var. Koreana NAKAI of the present invention can be prepared in two steps. First, a hot-water extract of dried Hovenia dulcis Thunb. var. Koreana NAKAI is obtained using a high pressure extraction procedure, and the hot-water extract thus obtained is treated with a lower alcohol to obtain said fraction.

For example, the fruit peduncle of Hovenia dulcis Thunb. var. Koreana NAKAI is sliced and dried in shades. Then, an appropriate amount of water is added to the dried slices and the mixture is kept at a temperature ranging from 110 to 150° C., preferably 120 to 125° C., under a pressure ranging from 1 to 3 atm., preferably 1.5 atm., for a period ranging from 15 min to 48 hrs, preferably 30 min to 12 hrs. Then the mixture is cooled to room temperature, filtered and the filtrate is lyophilized pursuant to a conventional lyophilizing method, to obtain a hot-water extract.

In the second step, the hot-water extract is further dried at room temperature, evaporated under a reduced pressure e.g., −1.5 atm, and extracted with a lower alcohol, e.g., methanol, ethanol or butanol, preferably 100% methanol, to remove lower alcohol-soluble components therefrom, to obtain the intended lower alcohol-insoluble fraction.

The lower alcohol-insoluble fraction thus obtained has the following characteristics: GPC (Gel Permeation Chromatography) peaks at mean M.W. of 1,330,000, 142,800, 70,540 and 102,400; IR (KBr, nm) absorption bands at 1000-1300 nm (ether, phenol, sulfoxide, vinyl peak); and a UV absorption at 200-300 nm (cyclic ring peak).

The lower alcohol-insoluble fraction contains a polysaccharide having a high hepatoprotective activity, which can be isolated by the following procedure.

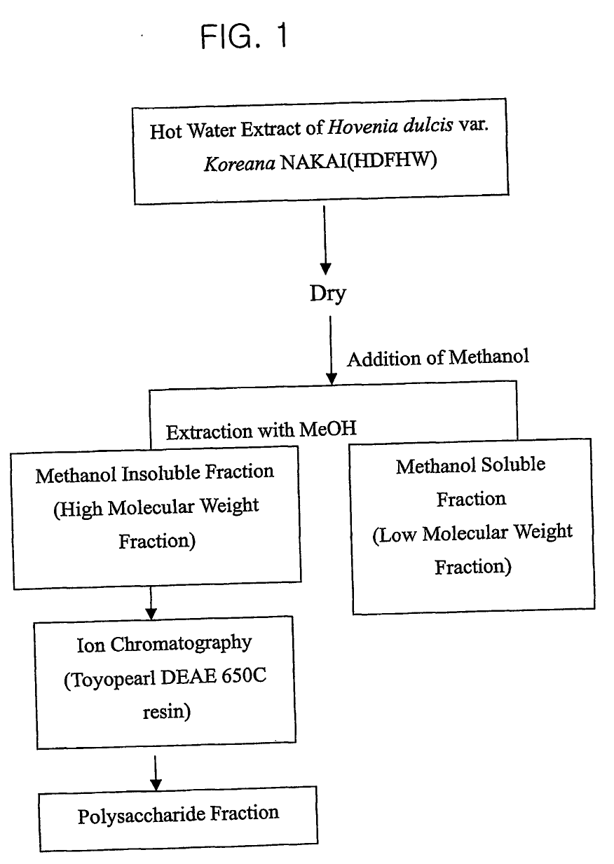

The lower alcohol-insoluble fraction is dissolved in distilled water, charged to an ion exchange column, eluted stepwise using solutions having increasing NaCl concentrations from 0 to 5M, dialyzed, concentrated, and lyophilized (see FIG. 1). In carrying out the ion exchange, either a cation exchange resin or an anion exchange resin may be used. Examples of exchange resins that can be used for this purpose are: strong acidic cation exchange resins such as AG 50W-x8, Amberlite IR-120, and Dowex 50W-x8; weak acidic cation exchange resins such as Amberlite IRC-50, Bio-Rex 70, Duolite-436; weak basic cation exchange resins such as Amberlite IRA-67, and Dowex 3-x4A; strong basic cation exchange resins such as AG 2×8, Amberlite IRA-400, and Dowex 2-x8; modified cellulose cation exchange resins such as CM-Celluose and SE-Cellulose; and anion exchange resins such as DEAE Celluose; cationic sephadex-type resins such as G-25 and G-50 bead type cross-linked dextran resins; and modified bead-type ion exchange resins made from agarose such as Cepharose CL, Biogel A Cepharose resin, Fractogels and Toyopearl. The preferred resins are Toyopearl DEAE type exchange resins, and the more preferred are Toyoprearl DEAE-650C type exchange resins.

Several polysaccharide fractions are obtained by the above isolation process and the polysaccharide that elutes with 0.2M NaCl shows the highest hepatoprotective activity. This polysaccharide shows the following characteristics: relative sugar contents calculated based on the amount of mannose (1%) are glucose (2.51%), galactose (12.53%), rhamanose (187%), and arabinose (13.43%); absolute molecular weight is 114,500; and IR (KBr, nm) shows peaks at 3550-3450 (broad, OH), 1660-1600(C═C), and 1290-1420(═CH—OH); and ¹H-NMR (600 MHz, D₂O) exhibits a peak at 4.4-4.8 ppm (sugar peak).

Various experiments clearly show that the lower alcohol-insoluble fraction and the polysaccharides isolated therefrom possess a high antihepatotoxicity and are effective for preventing and treating liver diseases.

Thus, the lower alcohol-insoluble fraction and the polysaccharide of the present invention may be employed as a pharmaceutical agent for preventing or treating liver toxicity and liver diseases such as hepatitis, fatty liver and liver cirrhosis.

Accordingly, the present invention also provides a pharmaceutical composition for inhibiting alcohol dehydrogenase and lactic acid dehydrogenase, which comprises the lower alcohol-insoluble fraction of Hovenia dulcis Thunb. var. Koreana NAKAI and the polysaccharide isolated therefrom as an active ingredient, in combination with pharmaceutically acceptable excipients, carriers or diluents. Additionally, the present invention provides a health care food comprising the extract and/or the polysaccharide described above.

The inventive pharmaceutical formulation may be prepared in accordance with any of the conventional procedures. In preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient. Thus, the formulation may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, or diluents are lactose, dextrose, sucrose, sorbitol, mannitol, starches, gum acacia, alginates, gelatin, calcium phosphate, calcium silicate, cellulose, methylcellulose, microcrystalline cellulose, polyvinylpyrrolidone, water, methylhydroxybenzoates, propylhydroxybenzoates, talc, magnesium stearate and mineral oil. The formulation may additionally include fillers, anti-agglutinating agents, lubricating agents, wetting agents, flavoring agents, emulsifiers, preservatives and the like. The composition of the invention may be formulated so as to provide a quick, sustained or delayed release of the active ingredient after it is administrated to a patient, by employing any one of the procedures well known in the art.

The pharmaceutical formulation of the present invention can be administered via various routes including oral, transdermal, subcutaneous, intravenous and intramuscular introduction. For treating a human patient, a typical daily dose of the above-mentioned fraction or polysaccharide isolated from Hovenia dulcis Thunb. var. Koreana NAKAI may range from about 0.01 to 10 g/kg body weight, preferably 1 to 5 g/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The above-mentioned lower alcohol-insoluble fraction and the polysaccharide isolated therefrom can be added to food or beverage for preventing various liver diseases and hangover. The amount of said fraction and/or polysaccharide that may be added to food or beverage for the purpose of preventing liver diseases may generally range from about 0.1 to 15 w/w %, preferably 1 to 10 w/w % based on the total weight of food, and 1 to 30 g, preferably 3 to 10 g based on 100 ml of beverage.

The health care beverage composition of the present invention may contain other components, e.g., deodorants and natural carbohydrates as in conventional beverages. Examples of such natural carbohydrates are monosaccharides such as glucose and fructose; disaccharides such as maltose and sucrose; conventional sugars such as dextrin and cyclodextrin; and sugar alcohols such as xylitol, sorbitol and erythritol. As the deodorant, a natural deodorant such as taumatin, levaudioside A, and glycyrrhizin, or a synthetic deodorant such as saccharin and aspartam may be used. The amount of the above-described natural carbohydrate is generally in the range of about 1 to 20 g, preferably 5 to 12 g based on 100 ml of beverage.

Other components that may be added to the inventive food or beverage composition are various nutrients, vitamins, minerals, synthetic flavoring agents, coloring agents pectic acid and its salt, alginic acid and its salt, organic acids, protective colloidal adhesives, pH controlling agents, stabilizers, preservatives, glycerin, alcohol, carbonizing agents, fruit juices and vegetable juices.

The following Reference Examples, Test Examples and Formulation Examples are intended to further illustrate the present invention without limiting its scope.

### REFERENCE EXAMPLE 1

### Determination of Absolute Molecular weight by Gel Permeation Chromatography

The molecular weight measurement was conducted using a GPC apparatus, equipped with a pump (spectra system, p2000 model), a guard column (TSK PWH, Tosoh Company), an RI-detector (Shodex SE71 model), SEC (size exclusion chromatography) columns (TSK gel 3000pw, 400pw, 5000pw (7.8×300 mm, Tosoh Company)), and a MALLS (multi angle laser light dispersion, Dawn DSP-F, Wyatt Technology Co.) detector, using a 0.02% sodium azide developing solvent containing 0.15 M NaNO₃ at a flow rate of 0.5 ml/min.

### REFERENCE EXAMPLE 2

### Reagents and Materials

The amount of LDH (lactic acid dehydrogenase) was determined with a 340-UV spectrometer (Sigma Co.). Both the 3H-Leucine (5μCi/plate) isotope used in determining the amount of the synthesized protein having healing activity of the liver damaged by a hepatotoxic substance and the 3H-Uridine isotope used in determining the amount of synthesized RNA were purchased from Sigma Co. The gas chromatography head space analytic method used for determining the activity of alcohol dehydrogenase by absorbance variation was conducted with HP 5890 gas chromatography (Hewlett Packard Company in USA) equipped with an FID (flame ionization detector).

### EXAMPLE 1

### Preparation a Lower Alcohol-Insoluble Fraction of Hovenia dulcis Thunb. var. Koreana NAKAI

Dried slices (1.5 kg) of the peduncle of Hovenia dulcis Thunb. var. Koreana NAKAI collected at the Korean National Arboretum site located at Chakdong-ri, Sohol-myun, Pochun-kun Kyunggi-do, were subjected to hot-water extraction at 120° C. for 3 hrs under a high pressure (1.5 atm) and the resulting extract solution was filtered through Wattman filtering paper. The filtrate (218.5 g) was lyophilized and the resulting powder was subjected to 3 cycles of reflux-extraction, each with 3 l of HPLC grade pure methanol for 1 hour. The extracts were combined and centrifuged for 10 to 20 min at 4000 rpm, to obtain a methanol-soluble fraction (dry weight 152.29 g, yield: 10.27% w/w) and a methanol-insoluble fraction (dry weight 65.71 g, yield: 4.3% w/w).

### EXAMPLE 2

### Analysis of the Methanol-Insoluble Fraction of Hovenia dulcis Thunb var. Koreana NAKAI

The properties of the methanol-insoluble fraction obtained in Example 1 were analyzed as follows.

(1) Determination of the Melting Temperature and Melting Entalphy

The melting temperature and melting entalphy were determined by DSC (Differential Scanning Calorimeter, Seiko Instruments Inc. DSC 6100). A sample of the methanol-insoluble fraction was placed in an aluminum pan, sealed, and then heated from 20° C. to 200° C. at a rate of 10° C./min, to obtain a melting heat absorption curve, and the melting temperature and crystalinity of the sample were determined therefrom.

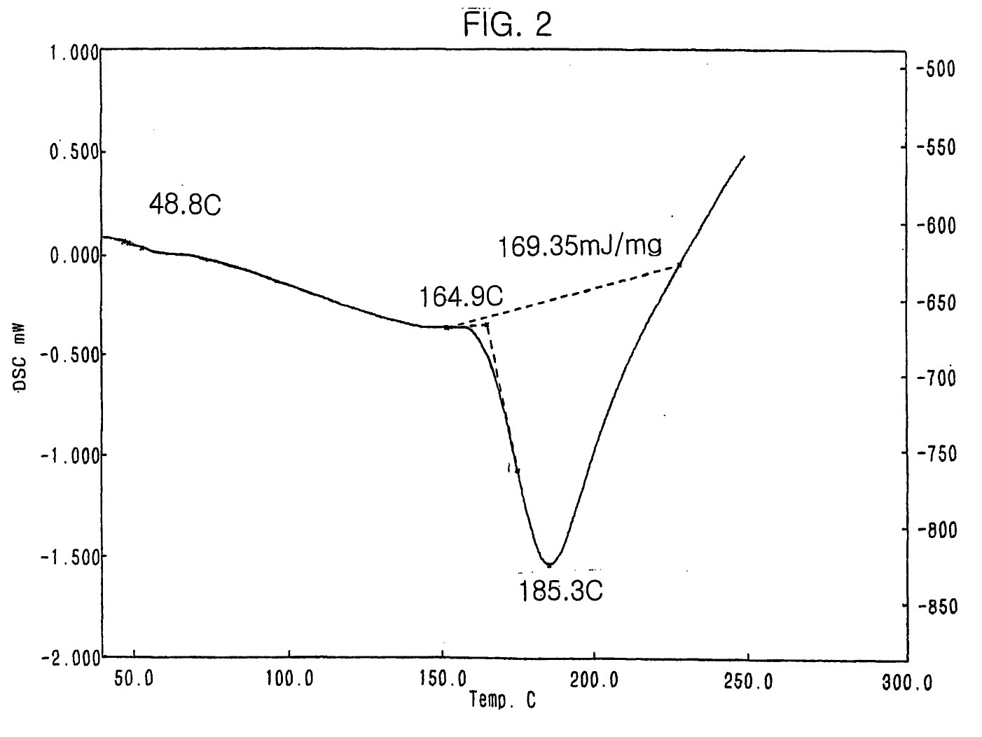

The DSC scan showed a main peak that started at 164.9° C. and reached the highest melting temperature at 185.3° C. The results suggested that the methanol-insoluble fraction contained several components: several carbohydrates (mp: 60˜100° C.) and minor amounts of proteins (mp: 60˜100° C.) (FIG. 2).

(2) Analysis for Sugar Chains

To examine whether the above main-peak components contained sugar chains, a methylation analysis was conducted according to the method described by Hakomori et al. (J. Biochem. Tokyo, 55, pp205-209, 1964) and Waeghe, T. J. et al. (Carbohydrate Research, 123, pp281-304, 1983).

A 500 μg sample was methylated, and then, the methylated product was collected using an ethanol-adsorbed C₁₈8×10 cartridge column (Sep-Pak). Acidic sugar moieties of the methylated product were reduced using LiB(C₂Hs)₃D (Super-Deupride, 1 ml, Aldrich Company) in THE and the reduction product was recovered using a C₁₈ 8×10 cartridge column (Sep-Pak). Subsequently, the treated sample was subjected sequentially to: hydrolysis at 121° C., for 2 hours in 1.0M TFA; reduction by NaBD₄; and acetylation. The resulting partially methylated alditol acetate was analyzed by GLC and GC-EIMS, and the peak areas were measured with an FID (flame ionization detector).

(3) Molecular Weight Determination by GPC

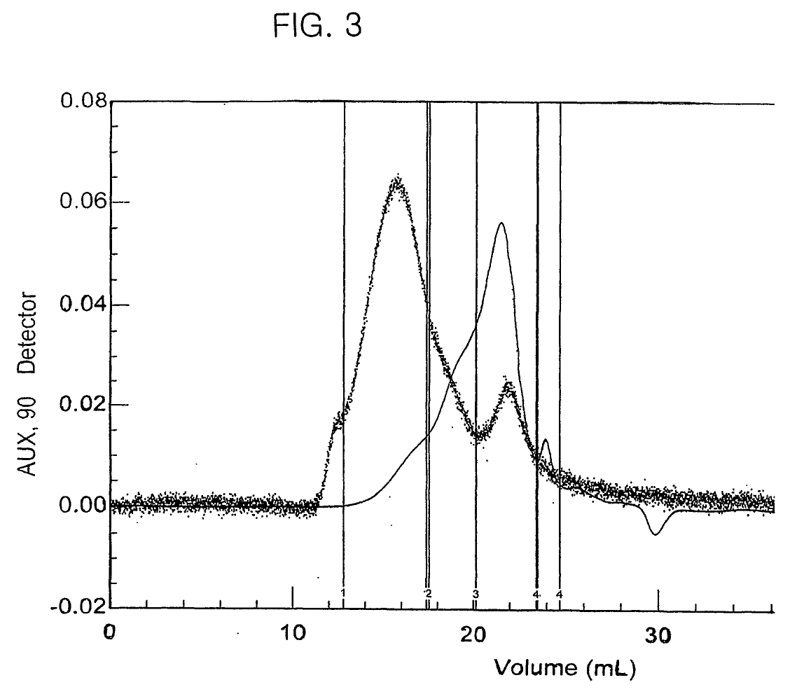

The result of a GPC conducted as in Reference Example 1 showed that the methanol-insoluble fraction was composed of 4 peak components as shown in FIG. 3 and in Table 1.

TABLE 1
Peak components of methanol-insoluble fraction

| Component | Peak 1 | Peak 2 | Peak 3 | Peak 4 |
|---|---|---|---|---|
| Mean MW | 1,330,000 | 142,800 | 70,540 | 102,400 |

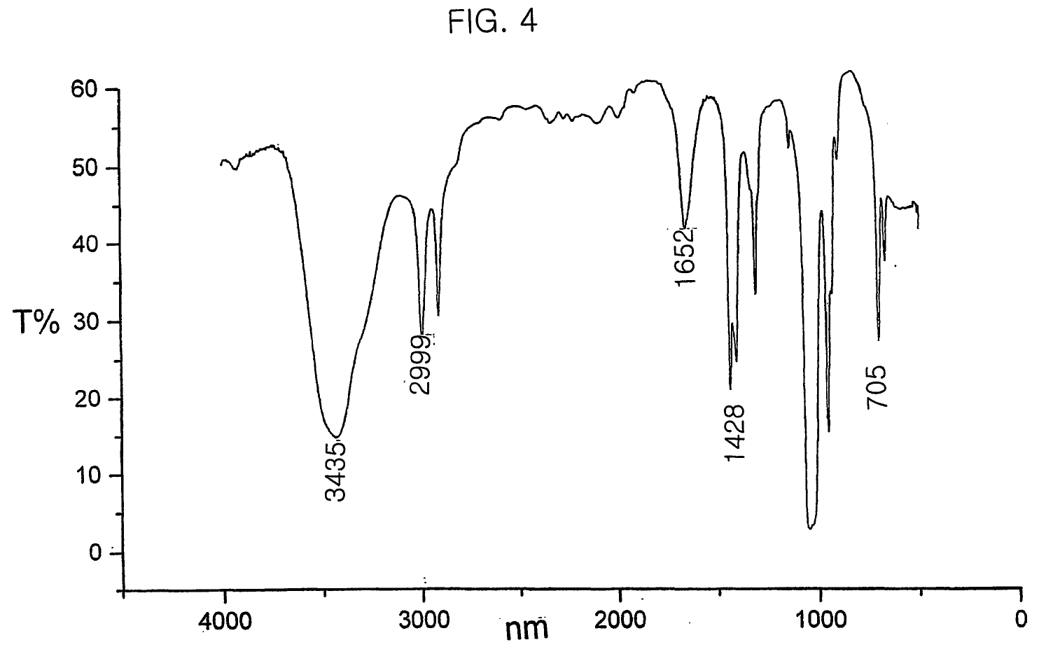

(4) IR Spectrum (FIG. 4)

The sample was analyzed by IR spectroscopy (Vector 22 model, Bruker Analytische Messtechnik GMBH): resolution, 4.0; source, sphere; velocity, 6, 10 KHz; capture mode, dual wall/forward-backward condition).

IR (KBr, cm⁻¹): ether, phenol, sulphoxide, vinyl peak (1000-1300 nm; main 1039 nm), aromatic ring peak (665, 939, 1313, 1663), hydroxyl peak (3435 nm).

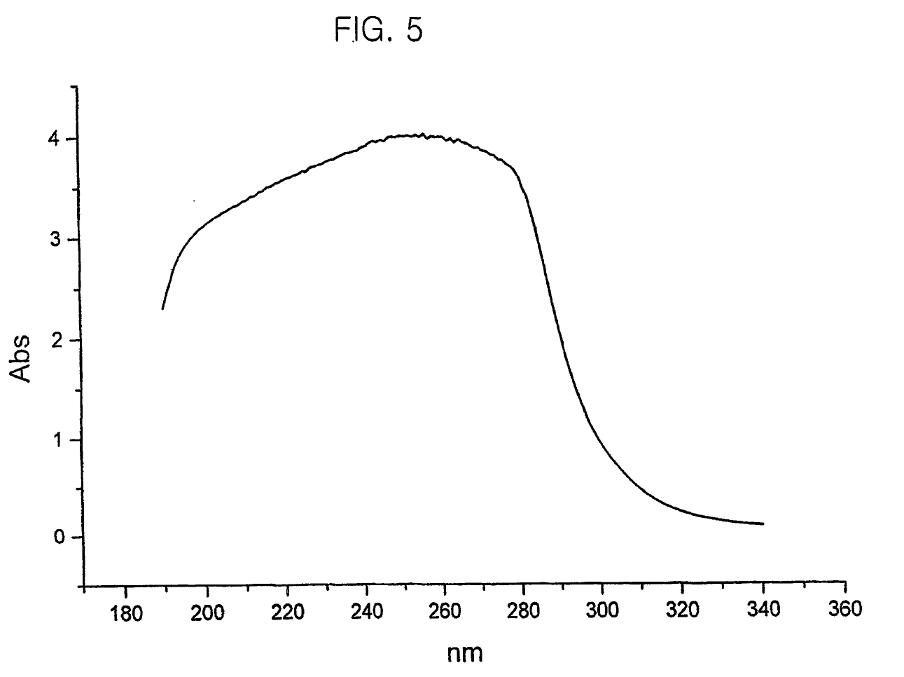

(5) UV Spectrum (FIG. 5)

The sample was analyzed by UV-Vis spectroscopy (HP 8453 model, Hewlett Packard Company) and the result showed a high absorbance at 200-300 nm, which suggested the existence of a cyclic ring.

### EXAMPLE 3

### Isolation of Polysaccharides having Hepatoprotective Activity

To isolate hepatoprotective active compounds from the methanol-insoluble fraction obtained in Example 1, 200 mg of the methanol-insoluble fraction was dissolved in distilled water, charged to a Toyopearl® DEAE-650C column (4.0×30 cm), and eluted successively with 0, 0.1, 0.2, 0.3 and 3M NaCl solutions. The eluted fractions were dialyzed using a dialysis membrane permeable at a M.W. of 1000 or below, concentrated, and then lyophilized to obtain purified fractions weighing 38 mg, 64 mg, 73 mg, 5 mg and 4 mg, respectively.

### Example 4

### Characterization of the Isolated Fractions

The isolated fractions obtained from Example 3 by eluting 0, 0.1, 0.2, 0.3 and 3M NaCl were designated as fractions 1 to 5, respectively. For each fraction, the amounts of total sugar and polyphenol components were determined by the phenol-sulfuric acid method (Dubois, M. et al.; Anal. Chem. 28, pp350-356, 1956), and the result thus obtained is shown in Table 2.

TABLE 2
Amounts of total sugar and polyphenol components

| Constituting | Amount (ppm) | | | | |
|---|---|---|---|---|---|
| Component | Fraction 1 | Fraction 2 | Fraction 3 | Fraction 4 | Fraction 5 |
| Carbo- | 248.79 | 231.21 | 122.76 | 51.81 | 2.33 |
| hydrate | | | | | |
| Polyphenol | 0 | 0.0245 | 0 | 0 | 0 |

GC analyses were conducted (Varian CP-3800 model, set-up condition; detector: FID, column: SP-2380 (30 m×0.25 mm×0.2 μm), temperature of column: 230° C., temperature of injector: 250° C., temperature of detector: 250° C., mobile phase: N₂ gas(1.0 ml/min)) to identify the sugar components of the above fractions and the relative amounts of mannose, glucose, ramanose, arabinose and xylose present in each fraction were determined. The result is shown in Table 3.

TABLE 3
Amounts of sugar components (relative to mannose)

| | | Glu- | Galac- | | | |
|---|---|---|---|---|---|---|
| | Mannose | cose | tose | Ramanose | Arabinose | Xylose |
| Fraction 1 | 1 | 6 | 3.1 | — | 1.58 | — |
| Fraction 2 | 1 | 3.29 | 2.04 | 0.32 | 1.93 | 0.26 |
| Fraction 3 | 1 | 2.51 | 12.53 | 187 | 13.43 | — |
| Fraction 4 | 1 | 1.13 | 7.81 | 3.64 | 5.86 | 0.75 |
| Fraction 5 | 1 | 3.05 | — | — | 1.93 | — |

Among the fractions, fraction 3 showed the highest hepatoprotective activity. Fraction 3 was further characterized as below.

Characterization of Fraction 3

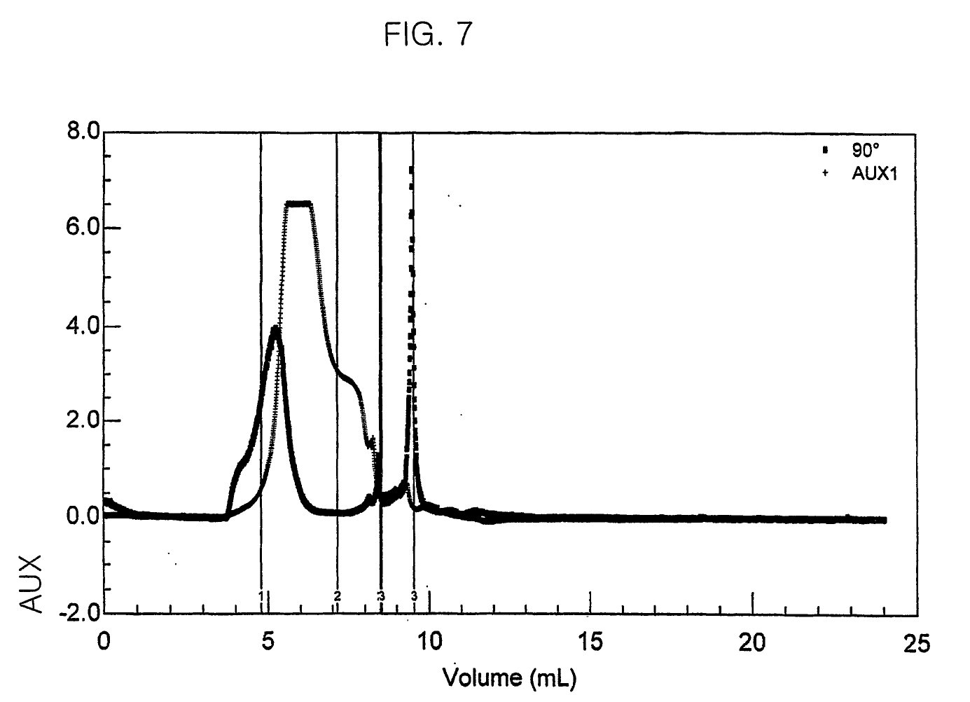

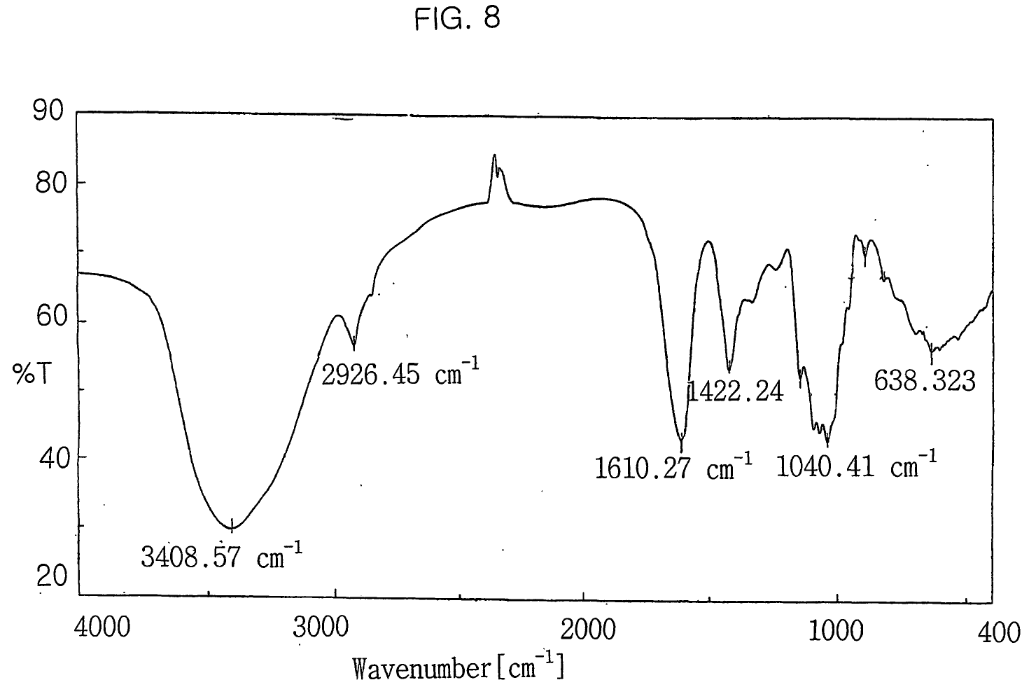

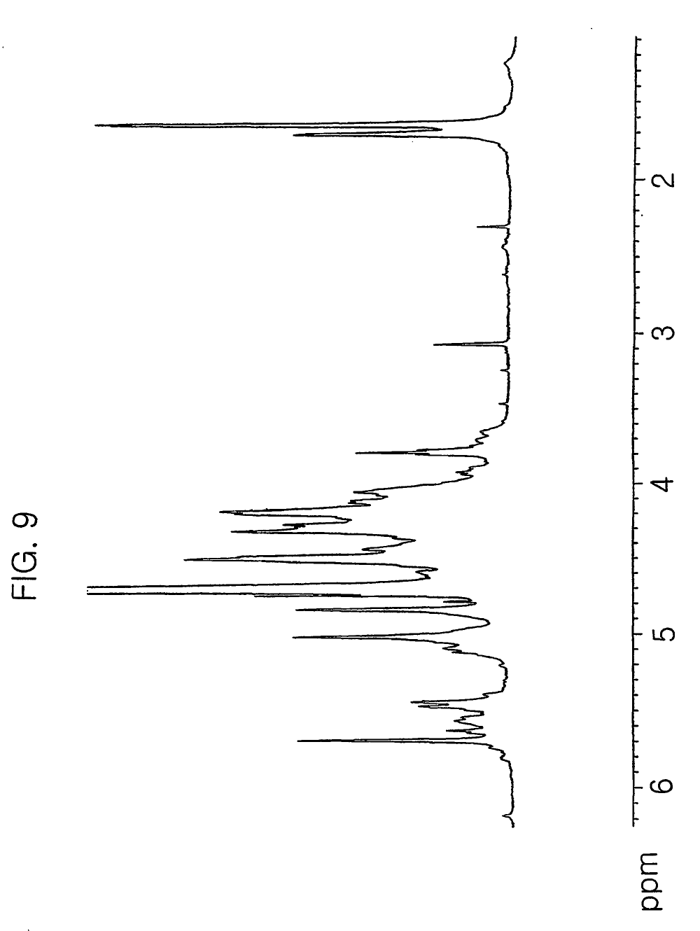

The absolute molecular weight of fraction 3 determined by the procedure of Reference Example 1 was 114,500 (FIG. 7). An IR analysis (KBr) showed peaks (cm⁻¹) at 3550-3450 (broad, OH), 1660-1600 (C═C) and 1290-1420 (═—CH—OH) (FIG. 8). ¹H-NMR (600 MHz, D₂O) showed a peak at 4.4-4.8 ppm (sugar peak) (FIG. 9).

### TEST EXAMPLE 1

### Hepatoprotective Activity of the Methanol-Insoluble Fraction of Hovenia duicis Thunb. var. Koreana NAKAI

(1) The Protective Activity of the Liver Damaged by Carbon Tetrachloride

Livers taken from five-week old Sprague-Dawley rats were sliced to obtain disc shaped samples each having a diameter of about 0.8 mm and a thickness of 200 μm (wet weight: 18-22 mg), using a tissue cutter (Brendel/Vitron Co., USA). The sliced samples were divided into 4 groups, two of which were treated with the methanol-insoluble fraction and the methanol-soluble fraction prepared in Example 1, respectively, each to a concentration of 200 μg/ml (group 3 and group 4, respectively). Then, the sliced sample were surface-cultured under an atmosphere of O₂/CO₂=95%/5% in a thermodynamic organ tissue cultivator (Sanyo Co., Japan). After 1 hour, carbon tetrachloride was added to a concentration of 4 mM to each of group 3, group 4 and one of the remaining two groups (group 2). The last remaining group (group 1) was treated with distilled water instead of carbon tetrachloride (control).

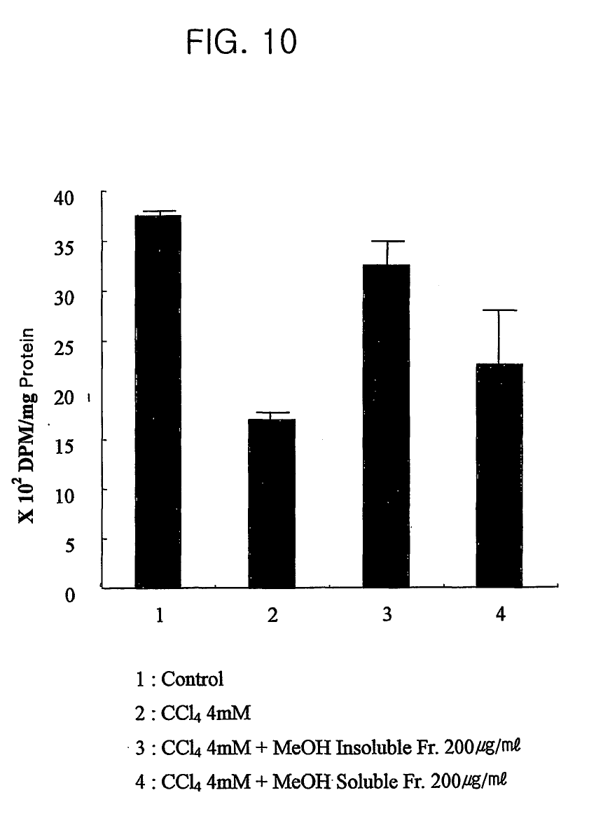

Thereafter, the liver detoxification efficacy of the extract fraction of Hovenia dulcis Thunb. var. Koreana NAKAI was evaluated by determining the amount of protein synthesized according to the method described by Bonney et al. (“Some Characteristics and Function of Adult Rat Liver Primary Culture, in Gene Expression and Carcinogenesis in Cultured Live”, (1975) Gerschenson, E and Thompson, E. B. (Eds), Academic Press, New York, pp2445). The result, shown in FIG. 10, demonstrates that the methanol-insoluble fraction has a much higher activity than the methanol-soluble fraction.

(2) The Activity for Restoring the Liver Function Damaged by D-galactosamine/LPS

It is known that D-galactosamine, when administrated together with bacterial lipopolysaccharide, causes liver damage, biochemically and histologically similar to that caused by human hepatitis. The detoxification effect of the methanol-insoluble fraction of Hovenia dulcis Thunb. var. Koreana NAKAI for such damaged liver was examined as follows.

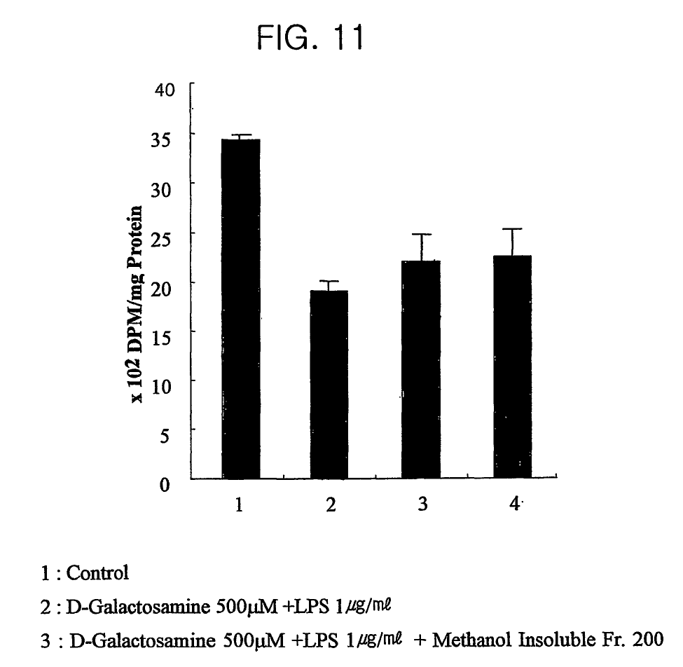

Instead of carbon tetrachloride, 500 μM D-galactosamine and 1 μg/ml LPS were used to repeat the procedure of Test Example 1. The result, shown in FIG. 11, shows that both of the methanol-insoluble and methanol-soluble fractions showed significant restoration activities.

(3) Restoring the Liver Damaged by Bromobenzene

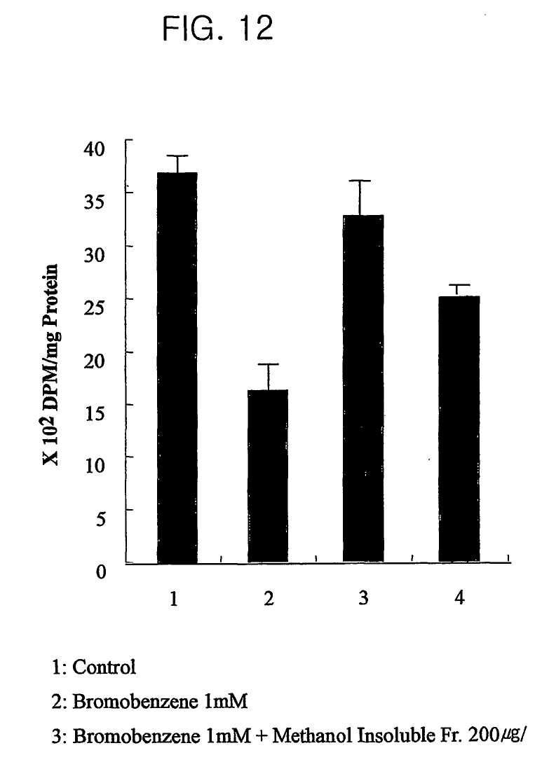

Except for using 1 mM of bromobenzene instead of carbon tetrachloride, the detoxification efficacy of the extract of Hovenia dulcis Thunb. var. Koreana NAKAI was evaluated by using the procedure of Test Example 1. The result, shown in FIG. 12, suggests that the methanol-insoluble fraction is more potent than the methanol-soluble fraction in restoring the damaged liver function.

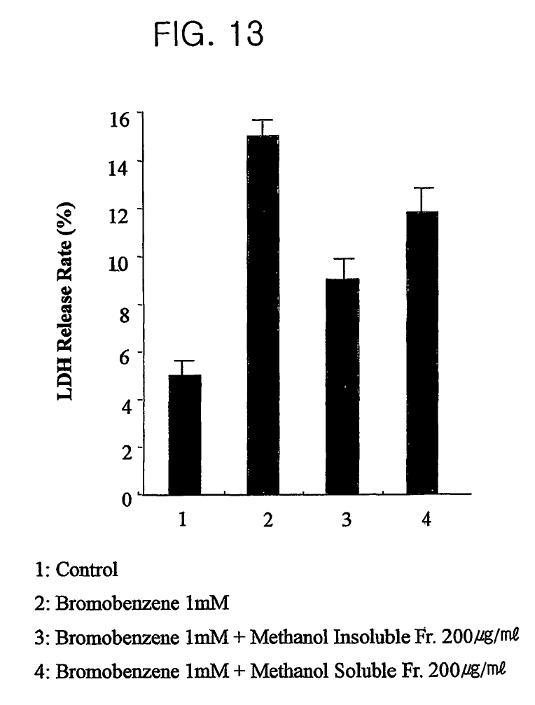

Additionally, the amount of LDH (lactic acid dehydrogenase) released from the culture medium was determined by using a Sigma kit 340-UV apparatus and the result showed FIG. 13) that the methanol-insoluble fraction is more potent than the methanol-soluble fraction in inducing the release of LDH caused by bromobenzene.

### TEST EXAMPLE 2

### Hepatoprotective activity of the polysaccharide isolated from the methanol-insoluble fraction of Hovenia dulcis Thunb. var. Koreana NAKA

Livers taken from five-week old Sprague-Dawley rats were sliced to obtain disc shaped samples each having a diameter of about 0.8 mm and a thickness of 200 μm (wet weight: 18-22 mg), using tissue cutter (Brendel/Vitron Co., USA). The sliced samples were divided into 7 groups, five of which were treated with polysaccharides prepared in Example 4, respectively, each to a concentration of 200 μg/ml (groups 1 to 5, respectively). Then, the sliced samples were surface-cultured under an atmosphere of O₂/CO₂=95%/5% in a thermodynamic organ tissue cultivator (Sankyo Co., Japan). After 1 hour, bromobenzene was added to a concentration of 4 mM to each sample of groups 1 to 5 as well as to one of the remaining two groups (group 6). The last remaining group (group 7) was treated with distilled water instead of bromobenzene (control).

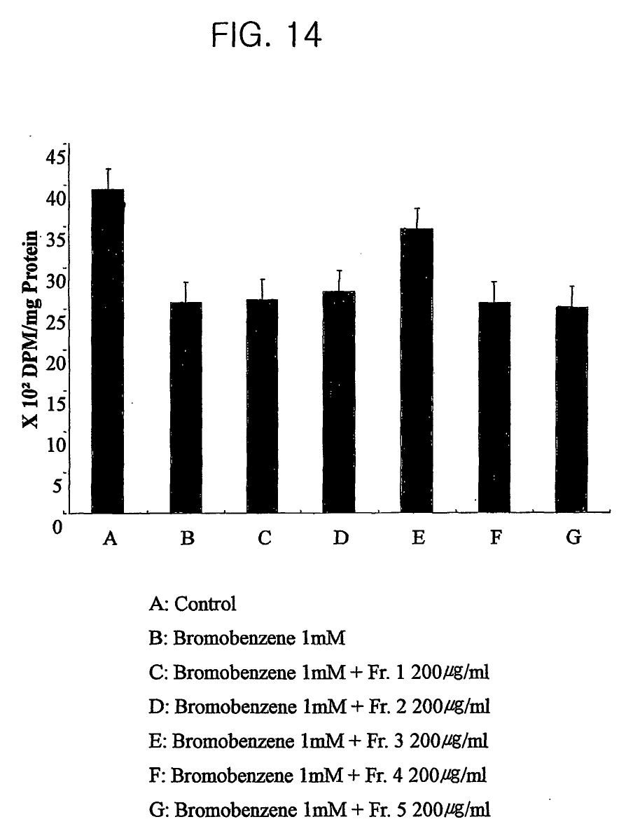

Thereafter, the liver detoxification efficacy of each polysaccharide fraction of Hovenia dulcis Thunb. var. Koreana NAKAI was evaluated by determining the amount of protein synthesized according to the method described by Bonney et al.(supra). The result, shown in FIG. 14, demonstrates that the polysaccharide of fraction 3 has the highest activity.

The lower alcohol-insoluble fraction and the polysaccharide isolated therefrom can be used in preparing a pharmaceutically effective powder, tablet, capsule, injection or liquid composition according to any one of the known conventional methods, as exemplified below.

FORMULATION EXAMPLE 1

2 g of the dried extract obtained in Example 1 was mixed with 1 g of lactose to obtain a powder preparation, which was filled and sealed in a sealed package.

### FORMULATION EXAMPLE 2

100 mg of the dried extract obtained in Example 1, 100 mg of corn starch, 100 mg of lactose, and 2 mg of magnesium stearate were mixed and tabletted to obtain a tablet preparation.

### FORMULATION EXAMPLE 3

100 mg of the dried extract obtained in Example 1, 100 mg of the corn starch, 100 mg of lactose, 2 mg of magnesium stearate were mixed and filled in a gelatin capsule to obtain a capsule preparation.

### FORMULATION EXAMPLE 4

100 mg of the dried extract obtained in Example 1, distilled water, and an appropriate amount of a pH controller were dissolved to obtain an injection formulation, which was filled in a 2 ml ample and sterilized according to a conventional injection preparation method, to obtain a injection preparation.

The heath care food was exemplarily prepared by the following method.

[Preparation of Health Care Food]

A scorched dried meal mixture of brown rice, barley, glutinous rice and Job's tear was pulverized and sieved to obtain grain particles of 60 mesh or less. Also, a mixture of black bean, black sesame and wild sesame was steamed, dried, scorched, pulverized and sieved to obtain seed particles of 60 mesh or less.

The dried methanol-insoluble fraction of Hovenia dulcis Thunb. var. Koreana NAKAI obtained in Example 1 was pulverized and sieved to obtain particles of 60 mesh or less, which were mixed with the grain particles and seed particles in the following proportions to prepare a granule type health food.

Grains: brown rice 30 w %, Job's tear 15 w % barley 20w %,

Seeds: wild sesame 7 w %, black bean 8 w %, black sesame 7 w %,

Dried powder of Hovenia dulcis Thunb. var. Koreana NAKAI: 3 w %

Shiitake mushroom 0.5 w %, rehmania root 0.5 w %

What is claimed is:

1. A lower alcohol-insoluble fraction of dried Hovenia dulcis Thunb. var. Koreana NAKAI.

2. The lower alcohol-insoluble fraction of claim 1, wherein the lower alcohol is selected from the group consisting of methanol, ethanol and butanol.

3. The lower alcohol-insoluble fraction of claim 2, wherein the lower alcohol is methanol.

4. A polysaccharide which is isolated from the lower alcohol-insoluble fraction of anyone of claims 1 to 3.

5. A process for preparing the lower alcohol-insoluble fraction of claim 1, 2 or 3, comprising the steps of: obtaining a hot-water extract of dried Hovenia dulcis Thunb. var. Koreana NAKAI; and subjecting the hot water extract to a lower alcohol extraction treatment to obtain the lower alcohol-insoluble fraction.

6. A process for preparing the polysaccharide of claim 4, comprising the steps of: obtaining a hot-water extract of dried Hovenia dulcis Thunb. var. Koreana NAKAI; treating the hot-water extract with a lower alcohol to obtain the lower alcohol-insoluble fraction; and subjecting the alcohol-insoluble fraction to an ion exchange column chromatography to obtain the polysaccharide.

7. The process of claim 5 or 6, wherein the lower alcohol is selected from the group consisting of methanol, ethanol and butanol.

8. The process of claim 7, wherein the lower alcohol is methanol.

9. The process of claim 6, wherein the ion exchange column chromatography is conducted using a cation exchange resin or an anion exchange resin.

10. The process of claim 9, wherein the ion exchange column chromatography is conducted using a modified bead-type ion exchange resin selected from the group consisting of Cepharose CL, Biogel A Cepharose resin, Fractogels and Toyopearl resin.

11. The process of claim 10, wherein the ion exchange column chromatography is conducted using the Toyopearl resin.

12. A composition for inhibiting alcohol dehydrogenase, comprising the lower alcohol-insoluble fraction of claim 1, 2 or 3 or the polysaccharide of claim 4, and a pharmaceutically acceptable carrier.

13. A pharmaceutical composition for preventing or treating a liver disease, comprising the lower alcohol-insoluble fraction of claim 1, 2 or 3 or the polysaccharide of claim 4, and a pharmaceutically acceptable carrier.

14. The pharmaceutical composition of claim 13, wherein the liver disease is hepatitis or liver cirrhosis.

15. A health care food comprising the lower alcohol-insoluble fraction of claim 1, 2 or 3 or the polysaccharide of claim 4, and a sitologically acceptable additive.

16. The health care food of claim 15, wherein the food is of a beverage type.
